(19) United States
(12) Patent Application Publication
(10) Pub. No.: US 2022/0028535 A1
(43) Pub. Date: Jan. 27, 2022

(54) METHOD FOR MITIGATING DISEASE TRANSMISSION IN A FACILITY

(71) Applicant: VergeSense, Inc., Mountain View, CA (US)

(72) Inventors: Dan Ryan, Mountain View, CA (US); Kelby Green, Mountain View, CA (US); Kanav Dhir, Mountain View, CA (US)

(21) Appl. No.: 17/386,226

(22) Filed: Jul. 27, 2021

Related U.S. Application Data
(60) Provisional application No. 63/057,251, filed on Jul. 27, 2020.
(60) Provisional application No. 63/197,922, filed on Jun. 7, 2021.

(51) Int. Cl. G16H 40/20; G06K 9/66; G06K 9/00

(52) U.S. Cl. CPC G16H 40/20; G06K 9/00476; G06K 9/00362; G06K 9/66

(57) ABSTRACT

A method for mitigating disease transmission in a facility includes: accessing a set of extant disease metrics associated with a reporting period; accessing a set of images of the facility captured during the reporting period by a set of sensor blocks deployed in the facility; aggregating the set of images into a timeseries of facility maps depicting the facility during the reporting period; identifying a set of objects in the timeseries of facility maps, the set of objects comprising a set of humans; generating a transmission feature vector based on the set of objects in the timeseries of facility maps and the set of extant disease metrics associated with the reporting period; calculating a predicted timeseries of health metrics for the facility based on the transmission feature vector and a facility health model; and prompting a mitigation response at the facility based on the predicted timeseries of health metrics.

### CROSS-REFERENCE TO RELATED APPLICATIONS

This application claims the benefit of U.S. Provisional Application No. 63/197,922, filed on 7 Jun. 2021, and U.S. Provisional Application No. 63/057,251, filed on 27 Jul. 2020, each of which is incorporated in its entirety by this reference.

This application is related to U.S. patent application Ser. No. 16/845,525, filed on 10 Apr. 2020, which is incorporated in its entirety by this reference.

### TECHNICAL FIELD

This invention relates generally to the field of occupancy monitoring and more specifically to a new and useful method for monitoring social distancing compliance in the field of occupancy monitoring

### BRIEF DESCRIPTION OF THE FIGURES

FIG. 1 is a flowchart representation of a first method;

FIG. 2 is a flowchart representation of a second method;

FIG. 3 is a schematic representation of a sensor block;

FIG. 4 is a flowchart representation of one variation of the first method; and

FIG. 5 is a flowchart representation of one variation of the first method.

### DESCRIPTION OF THE EMBODIMENTS

The following description of embodiments of the invention is not intended to limit the invention to these embodiments but rather to enable a person skilled in the art to make and use this invention. Variations, configurations, implementations, example implementations, and examples described herein are optional and are not exclusive to the variations, configurations, implementations, example implementations, and examples they describe. The invention described herein can include any and all permutations of these variations, configurations, implementations, example implementations, and examples.

### 1. Method for Mitigating Disease Transmission in a Facility

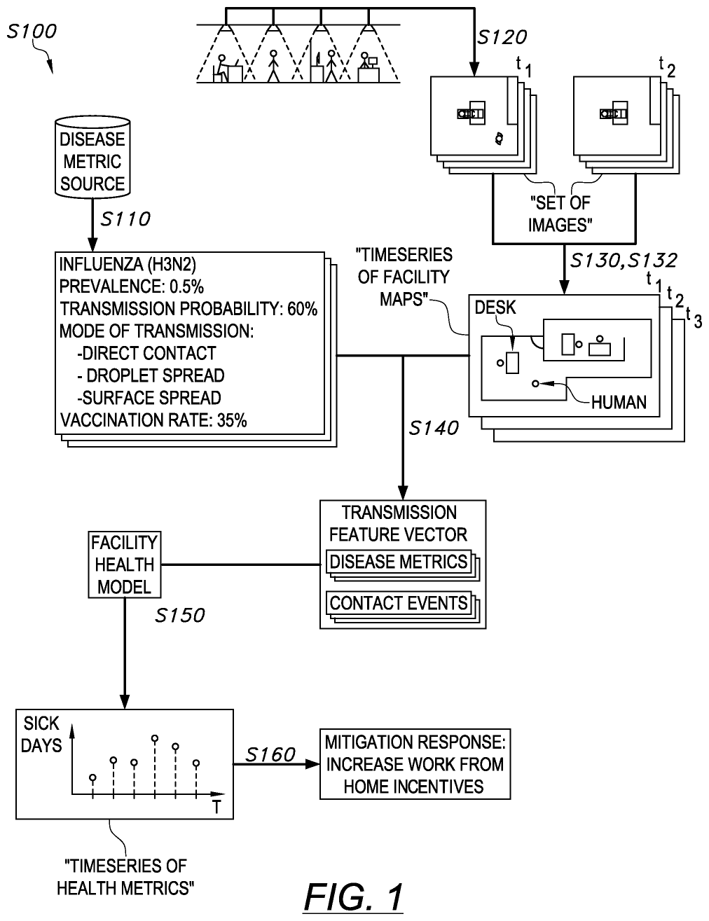

As shown in FIG. 1, a method S100 for mitigating disease transmission in a facility includes: accessing a set of extant disease metrics associated with a reporting period in Block S110; accessing a set of images of the facility captured during the reporting period by a set of sensor blocks deployed in the facility in Block S120; aggregating the set of images into a timeseries of facility maps depicting the facility during the reporting period in Block S130; identifying a set of objects in the timeseries of facility maps, the set of objects comprising a set of humans in Block S132; generating a transmission feature vector based on the set of objects in the timeseries of facility maps and the set of extant disease metrics associated with the reporting period in Block S140; calculating a predicted timeseries of health metrics for the facility based on the transmission feature vector and a facility health model in Block S150; and prompting a mitigation response at the facility based on the predicted timeseries of health metrics in Block S160.

### 2. Method for Training a Facility Health Model

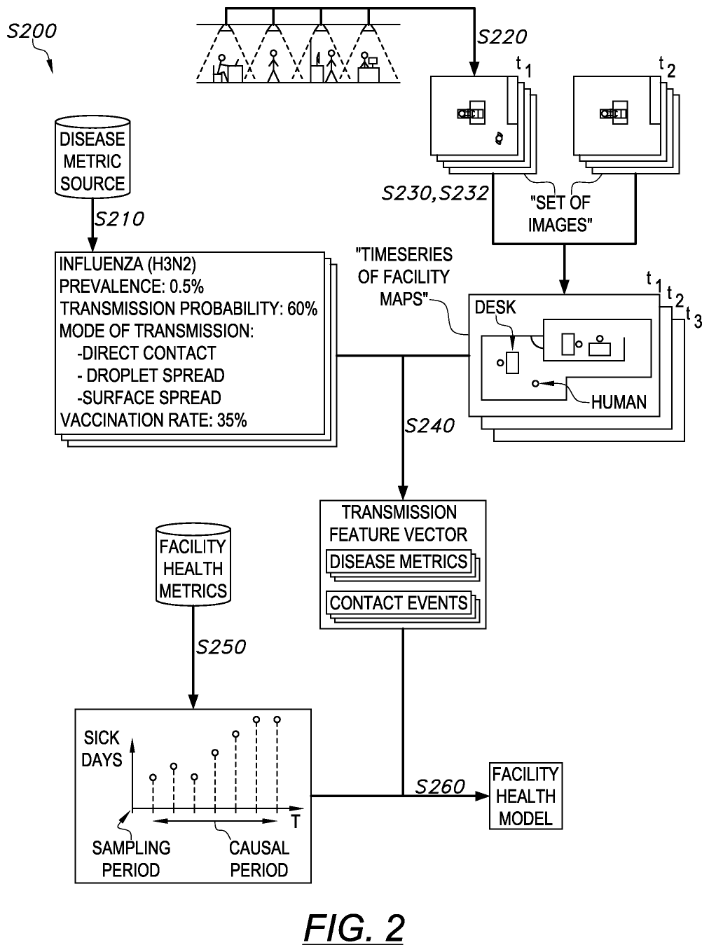

As shown in FIG. 2, a method for training a facility health model includes, during a training period and for each sampling period in the training period: accessing a training set of extant disease metrics associated with the sampling period in Block S210; accessing a training set of images of the facility captured during the sampling period by a set of sensor blocks deployed in the facility in Block S220; aggregating the training set of images into a training timeseries of facility maps depicting the facility during the sampling period in Block S230; identifying a training set of objects in the timeseries of facility maps, the training set of objects comprising a training set of humans in Block S232; generating a training transmission feature vector based on the training set of objects in the training timeseries of facility maps and the training set of extant disease metrics associated with the sampling period, the training transmission feature vector in Block S240; and accessing a timeseries of health metrics for a causal period subsequent to the sampling period in Block S250. The method S200 also includes training the facility health model based on: the training transmission feature vector for each sampling period in the training period; and the timeseries of health metrics for the causal period subsequent to each sampling period in the training period in Block S260.

### 3. Applications

Generally, as shown in FIG. 1, the method S100 can be executed by a computer system (hereinafter “the system”) in association with a facility—such as an office building, a school, a retail environment, a transportation hub, or any other facility—and in cooperation with a set of sensor blocks deployed throughout the facility in order to mitigate disease transmission among humans within the facility. More specifically, the system can, based on images captured by the set of sensor blocks: detect probable transmission of disease in the facility; predict health outcomes for humans in the facility; and automatically prompt mitigation responses for the facility. In particular, the system can: access a set of images of the facility over a reporting period; generate a timeseries of facility maps for the reporting period, such that each facility map represents the locations of humans in the facility; generate a transmission feature vector based on the timeseries of facility maps; input this transmission feature vector into a facility health model to predict a timeseries of health metrics for the facility; and prompt a mitigation response based on the timeseries of health metrics. Thus, the system can automatically adjust parameters—such as a proportion of employees at the facility working from home, the maximum number of simultaneous occupants allowed within a conference room or agile desk area, the density and/or placement of agile desks deployed within the facility, vacation or paid-time-off incentives for employees, and/or the social distancing policy within the facility—to reduce instances of disease transmission within the facility and therefore improve the health status and productivity of humans occupying the facility.

Additionally, the system can incorporate real-time or near real-time extant disease data when predicting health outcomes for humans within the facility. More specifically, the system can access a repository for communicable or transmissible diseases (e.g., bacterial, viral) spreading within a local population of the facility. For example, the system can access extant disease data indicating the prevalence of a new disease in the local population, its mode of transmission (e.g., direct contact, droplet spread, airborne spread, vehicle-borne, vector-borne), its probability of transmission given sufficient contact with an infected individual, and its health impact on an individual given infection. Thus, the system can: adjust the aforementioned parameters at a facility to reduce disease transmission while the risk of negative health impacts from a disease is high; and relax these mitigation measures while the risk of negative health impacts is low.

By predicting a timeseries of health metrics for humans within the facility, the system can also provide to administrators of the facility valuable data with which to plan future actions within the facility. For example, the system can predict that fifteen employees are likely to be symptomatic with a rhinovirus within the next five days. In this example, an administrator can then react to the prediction output by the system by hiring temporary contractors for the subsequent five days. Thus, by predicting a timeseries of specific health outcomes for the population of humans within the facility, the system enables administrators to mitigate the effect of illness on a business operating within the facility.

Furthermore, the system can also predict health outcomes and prompt corresponding mitigation responses based on the localized context provided by the timeseries of facility maps. For example, the system can detect, via the timeseries of facility maps, that 70% of contact events between humans in the facility occur in the cafeteria, 20% in the agile desks and 10% in conference rooms. The system can then prompt a localized mitigation response by prompting closure of the cafeteria, thereby reducing future contact events by 70%. Thus, the system can provide location specific mitigation responses within a facility to enable humans to occupy the facility without risking undue exposure to extant diseases.

As shown in FIG. 2, the system can execute Blocks of the method S200 to train the facility health model based on a corpus of data collected over a training period. More specifically, the system can: access a training set of images of the facility over a sampling period within the training period; generate a training timeseries of facility maps for the sampling period; generate a training transmission feature vector based on the training timeseries of facility maps; access a timeseries of health metrics for humans in the facility for a causal period subsequent to the sampling period; and train the facility health model based on the training transmission feature vector and the timeseries of health metrics subsequent to the generation of this feature vector. Thus, the system can execute supervised learning algorithms to increase the accuracy of the facility health model over time, thereby improving the efficacy of mitigation responses prompted via Blocks of the method S100.

The system can also detect a level of compliance with a social distancing policy by humans in the facility; report this level of compliance to administrators of the facility; and/or automatically update facility schedules or asset distributions—via a cooperating scheduler or asset management application—to reduce the probability of future non-compliance within the facility.

In order to detect distances between humans within the facility, the system can capture images (e.g., overhead images) of a floor area (e.g., a room or region) within the facility; analyze these images to detect positions of humans, assets (e.g., chairs, desks), and obstructions within the facility; and measure the distances between these positions via photogrammetry techniques. For each image, the system can measure the distance between humans in the image (e.g., a point-to-point distance between reference positions corresponding to these humans) on a pairwise basis. The system can then aggregate these distances over multiple successive images recorded at many sensor blocks to generate an overarching summary of social distancing compliance within the facility.

In one variation, the system can, upon detecting a pair of humans in sufficiently similar positions (e.g., within a threshold distance of a position of a human in a prior image) across multiple successive images, calculate a contact duration for the interaction between this pair of humans in order to characterize potentially infectious contact events within the facility according to time as well as distance.

While aggregating social distancing data across successive images captured by the set of sensor blocks, the system can generate real-time alerts or notifications (at an administrative portal or application) to administrators of the facility in order to notify these administrators of ongoing non-compliance with a social distancing policy. The administrators of the facility may then approach those humans not complying with the social distancing policy of the facility and request compliance with the social distancing policy. Alternatively, the system can aggregate the social distancing data over time and periodically generate a report to administrators of the facility indicating the level of compliance with social distancing policies within the facility over a time period (e.g., a day, a week, a month). The system can display the social distancing data as a set of summary metrics, as a distribution of distances between humans, and/or as a heatmap indicating locations within the facility where frequent interactions between humans and/or frequent violations of the social distancing policy occurred during the time period. Thus, the system can communicate an up-to-date level of compliance with the social distancing policy to administrators of the facility and facilitate identification of environmental factors within the facility that may be contributing to non-compliance with the social distancing policy of the facility.

In addition to notifying administrators of the facility regarding social distancing compliance, the system can also automatically reschedule meetings, redirect assets, and/or schedule cleanings based on patterns in the social distancing data. For example, the system can detect a concentration of individuals within a conference room and, in response, schedule a cleaning for this conference room later in the day. Alternatively, in an example in which the system cooperates with an agile desk manager (i.e., an application that reserves or schedules occupancy of agile desks within a workplace), the system can, in response to detecting a threshold number of social distancing policy violations within the agile desk area, disable reservations for desks within a threshold distance of previously reserved desks. In another example, the system can, in response to detecting a threshold number of social distancing policy violations within a conference room, generate a work order to remove chairs from the conference rooms to discourage overcrowding within the conference room. Thus, the system can aid and/or guide administrators in order to reduce exposure of communicable diseases to humans within the facility.

### 4. System

Generally, the system can include a computational device, such as a server or set of remote servers, configured to execute Blocks of the methods S100 and/or S200 (although each of these methods can also be executed by independent servers or sets of servers). The system can cooperate with: a set of sensor blocks deployed throughout the facility and configured to periodically record images (e.g., overhead images) of the facility; a set of local gateways arranged throughout the facility and configured to pass data between these sensor blocks and the remote server; and/or a scheduler system that serves prompts to administrators of the facility. Thus, in some implementations, select Blocks of the methods S100 and/or S200 can be executed by the set of sensor blocks, the set of local gateways, and/or the scheduler system at the facility.

### 4.1 Sensor Blocks

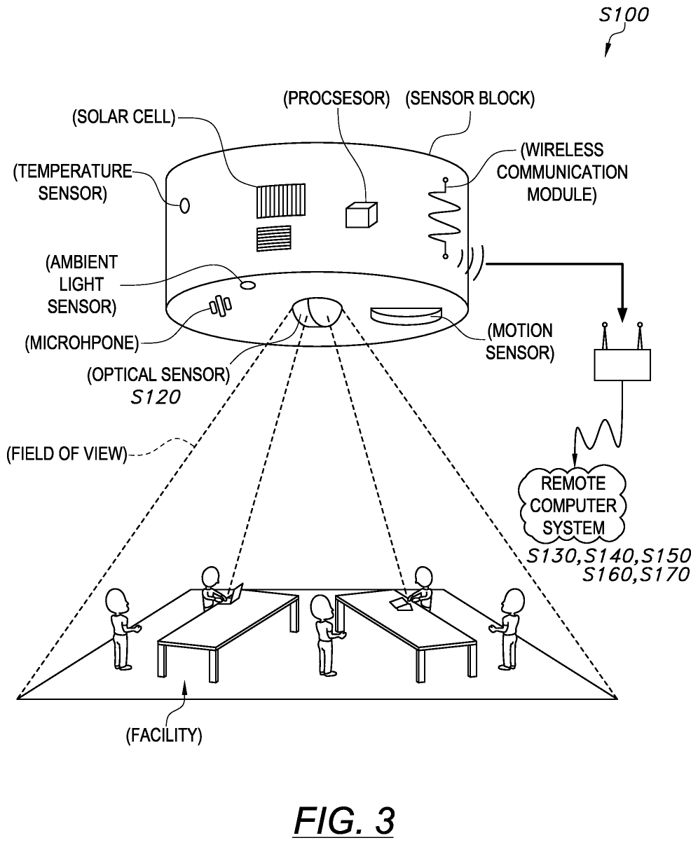

As shown in FIG. 3, the system can include a set of sensor blocks similar to those described in U.S. patent application Ser. No. 16/845,525 and deployed within the facility as described in U.S. patent application Ser. No. 16/845,525. Thus, each sensor block in the set of sensor blocks can capture a set of images of a floor area within the field of view of the sensor block and detect humans and/or other objects occupying this floor area.

### 5. Reporting Period

Generally, the system can periodically (e.g., once per reporting period) execute Blocks of the method S100 to improve health outcomes for a set of humans within the facility—such as employees of the facility, attendees of the facility, the local population (for a public facility), or any combination of humans likely to be present within the facility. More specifically, the system can: access extant disease metrics and a set of images of the facility during the reporting period; and predict health outcomes based on these data collected during the reporting period. In one implementation, the system operates on a reporting period of one day. Therefore, the system accesses the extant disease metrics for the day and collects a set of images of the facility captured by the set of sensor blocks during the same day. At the end of the day, the system can then: generate the transmission feature vector; predict health metrics for a time period subsequent to the day; and prompt a mitigation response for execution subsequent to the day. The system can execute Blocks of the method S100 based on a reporting period of one hour, one day, one week, one month, or any other time period according to the needs of an administrator of the facility.

In one implementation, the system can access extant disease metrics and/or a set of images from multiple consecutive reporting periods (i.e., a sliding buffer of reporting periods) in order to improve health metric prediction and disease mitigation. In this implementation, the system can utilize extant disease metrics and a set of images from multiple reporting periods when generating the transmission feature vector, as is further described below.

### 6. Sampling Period

Generally, the system can periodically (e.g., once per sampling period) execute Blocks of the method S200 during a training period in order to train a facility health model for execution of Block S150 of the method S100. More specifically, the system can select a sampling period equal to the reporting period to ensure that training transmission feature vectors generated in Block S240 are representative of transmission feature vectors generated in Block S140. Thus, the system can select a sampling period according to any of the implementations described above with respect to the reporting period.

### 6.1 Causal Period

Generally, in Block S260 the system executes a supervised learning method to train the facility health model based on a set of training examples, each training example including a training transmission feature vector generated based on data from a sampling period and a timeseries of health metrics for a causal period subsequent to the sampling period. The system can select this causal period based on the maximum total duration of an incubation period and a clinical period for an extant disease to be tracked by the system. For example, the system can be configured to predict health outcomes related to coronavirus disease 2019 (hereinafter, “COVID-19”), which has an incubation period of up to 14 days and a clinical period of up to 90 days (with high variation). Thus, in this example, the system can select a causal period of 104 days (i.e., the sum of the incubation and clinical period) for training the facility health model to account for the full progression of COVID-19. However, the system can select a causal period based on any other metric associated with an extant disease to be tracked by the system via Blocks of the method S100.

### 7. Extant Disease Metrics

Generally, the system can access a set of extant disease metrics associated with a reporting period in Block S110 and/or associated with a sampling period in Block S210, as is further described below. More specifically, the system can access extant disease metrics for each extant disease in a set of extant diseases. For example, the system can access extant disease metrics for the common cold, two different flu strains, strep throat, and COVID-19. Thus, by accessing a set of extant disease metrics, the system can predict and subsequently mitigate the effects of multiple extant diseases simultaneously.

In one implementation, the system can access the set of extant disease metrics from a trusted private or governmental disease tracking source (e.g., the Center for Disease Control, hereinafter, “CDC,” in the United States, the World Health Organization, hereinafter, “WHO”). In another implementation, the system can initially access the set of extant disease metrics from a trusted disease tracking source and subsequently update these extant disease metrics based on observations within the facility. For example, the system can update the prevalence of COVID-19 in the set of extant disease metrics based on a known number of COVID-19-positive individuals within the facility. In yet another implementation, the system can estimate the set of extant disease metrics based on known seasonal patterns for specific diseases (e.g., common colds, allergies). However, the system can access and/or update any subset of extant disease metrics using any combination of the above-described implementations of Blocks S110.

Generally, the system can access a set of extant disease metrics for each reporting period or sampling period for incorporation into the transmission feature vector or training transmission feature vector, respectively. For example, if the system is executing the method S100 according to a reporting interval of one day, the system can update the set of extant disease metrics on a daily basis. Thus, the set of extant disease metrics included in the transmission feature vector are relevant to the specific reporting period for which the system is executing Blocks of the method S100 or S200.

The system can access a set of extant disease metrics for each extant disease in the set of extant diseases, such as a prevalence of the extant disease, a mode of transmission of the extant disease, a probability of transmission of the extant disease, and a health impact of the extant disease. Thus, the system can, via the facility health model, correlate particular characteristics of the set of extant diseases relevant to the facility to particular health outcomes for the set of humans of the facility, thereby increasing the accuracy of these predictions as the set of extant diseases changes over time.

In one implementation, the system can access a prevalence of an extant disease in the set of extant disease metrics such that the prevalence of the extant disease represents the likelihood that a human in the facility is infected with the extant disease and is currently contagious. Thus, the system can estimate a prevalence of an extant disease based on a known infection rate of the disease in a local population relevant to the facility, the average (or median) subclinical infectious period of the extant disease, the average (or median) infectious period of the extant disease, the attendance impact of the disease (e.g., whether the clinical signs of the disease are likely to prevent an infected human from attending the facility), and/or the growth rate of the extant disease in the local population. For example, if the infection rate of the extant disease is 5%, the subclinical infectious period is four days, the infectious period is eight days, the attendance impact is 40% (e.g., 40% of people with clinical signs do not attend the facility), and the growth rate of new infections is steady (i.e., 1.0), the system can estimate a prevalence of the disease in the facility at 4% (i.e., ( 4/8*0.05*1.0)+( 4/8*0.05*(1−0.40))).

In another implementation, the system can access a mode of transmission of an extant disease in the set of extant disease metrics. In this implementation, the mode of transmission of an extant disease is a categorical variable indicating the mechanism by which the disease is transmitted between humans. The system can access modes of transmission for extant diseases including: direct contact, droplet spread, airborne spread, vehicle-borne, vector-borne. Thus, the system can model (via the facility health model) the probable transmission of the extant disease according to the physical mode of transmission of the extant disease. In one implementation, the system can define a contact event for inclusion in the transmission feature vector based on the mode of transmission, further described with respect to Block S140 below.

In yet another implementation, the system can access a probability of transmission of an extant disease in the set of extant disease metrics. The probability of transmission indicates the probability that, given a contact event (e.g., defined by the mode of transmission), the disease spreads between the infected human and the non-infected human. Thus, the system can account for (via the facility health mode) the transmissibility of each extant disease represented in the set of extant disease metrics.

In yet another implementation, the system can access a vaccination rate of an extant disease in the set of extant disease metrics. The vaccination rate of an extant disease indicates the prevalence of vaccinated humans among the population of humans in the facility or likely to be in the facility (e.g., based on the vaccination rate of a local population of humans). Additionally, the system can access an immunity rate for an extant disease indicating both the proportion of humans that have been vaccinated and the proportion of humans that have already been infected with the extant disease but are no longer symptomatic. Thus, the system can account for immunity in the population of humans within the facility when predicting health outcomes for the population of humans in the facility.

### 8. Image Capture

Generally, the system can access a set of images of the facility captured during the reporting period by a set of sensor blocks deployed in the facility in Block S120 or captured during a sampling period in Block S220. More specifically, each sensor block in the set of sensor blocks can, for each scan cycle during the reporting period: capture an image in the set of images, the image depicting the facility with a field of view of the sensor block; and transmit the image to a remote server executing the method S100 or S200; and, at the remote server, access the set of images. The set of sensor blocks can transfer the set of images to the remote server via a set of gateways distributed throughout the facility. Thus, the system can receive images from multiple locations in the facility in near real-time and aggregate these images to obtain a full representation of the positions and/or orientations of objects and humans within the facility.

In one implementation, the set of sensor blocks are configured to execute a scan cycle on a preset interval (e.g., one minute, five minutes, ten minutes). Alternatively, the set of sensor blocks can execute scan cycles on a variable interval based on detected movement within or near to the field of view of each sensor block (e.g., in order to conserve battery life of the sensor block). Thus, the set of sensor blocks captures a set of images depicting various locations across the facility over a series of scan cycles spanning the reporting period (or sampling period during execution of the method S200).

In another implementation, the set of sensor blocks capture images during synchronous scan cycles. More specifically, the set of sensor blocks are configured to capture images approximately simultaneously (e.g., within a five-second time window), thereby preventing double counting of humans or assets within the facility and enabling the system to generate an accurate representation of the entire facility for each synchronous scan cycles of the set of sensor blocks. Alternatively, the set of sensor blocks can capture images during asynchronous scan cycles and the system can: define a set of time windows within the reporting period or sampling period; and aggregate a subset of images, in the set of images, captured during each time window in order to generate multiple representations of the facility over time.

In yet another implementation, the set of sensor blocks captures the set of images as a set of continuous video feeds (e.g., with less the one second between frames). In this implementation, the system can access these video feeds and generate a timeseries of facility maps with a greater resolution, thereby improving tracking and path identification of humans in the facility and further increasing the accuracy of the predicted health metrics and mitigation responses generated via execution of Blocks of the method S100.

### 9. Facility Maps

Generally, the system can aggregate the set of images into a timeseries of facility maps depicting the facility during the reporting period in Block S130 or during the sampling period in Block S230. More specifically, the system can generate a timeseries of facility maps depicting locations of a set of objects within the facility during the reporting period based on the set of images, the set of objects including a set of humans. Thus, the system can generate a timeseries of facility maps that indicates the location of each object in the set of objects within the facility and enables identification of contact events between humans in the facility.

In one implementation, the system generates facility maps that each represent the location and identity of each object detected in the set of images during a particular time window corresponding to the facility map. For example, the system can generate a facility map for a ten-second time window within the reporting period based on a subset of images in the set of images captured during that ten-second time window. In one example, the system can generate a facility map that indicates the location of each object based on a two-dimensional coordinate space. In this example, the system can map specific regions of the facility within the fields of view of the set of sensor blocks (which may be non-contiguous based on the distribution of sensor blocks in the facility) onto the two-dimensional coordinate space and label objects detected in the set of images based on this mapping. Alternatively, the system can identify the height or vertical position of each object and represent the location of the object within a three-dimensional coordinate space. Additionally, the system can label each object based on a classification or classification vector output by an object detection model in order to identify the object in the facility map. Thus, each facility map can be represented as a list of objects identified by classification and located within a coordinate system mapped onto the facility.

The system can generate a facility map that is anonymized and does not include personally identifiable information. For example, by representing objects in the facility map based on a categorical classification (e.g., human, chair, computer, bag) or classification vector, the system generates a facility map that does not include any underlying image data in the set of images from which the system generated the facility map.

In one implementation, in order to generate a facility map, the system can: aggregate overlapping images in the set of images (e.g., via image stitching) captured within a time window in the recording period or sampling period; and execute an object identification model on the aggregate image depicting the facility (the image may include discontinuities where the fields of view of the set of sensor blocks do not overlap). Alternatively, the system can: execute the object identification model on the set of images to generate a set of feature-based images; and aggregate these feature-based images into the facility map. In this alternative implementation, the system can aggregate the feature-based images into the facility map by combining like features (i.e., detected objects) within regions corresponding to overlapping fields of view of two or more sensor blocks in the set of sensor blocks. Thus, the system can generate a facility map by aggregating the set of images for particular time windows of the reporting period or sampling period and identifying objects within these images either before or after aggregation of these images.

In another implementation, the system can generate a timeseries of facility maps including the air circulation of each region of the facility (e.g., based on a schematic of the heating ventilation and air conditioning system, hereinafter, “HVAC system,” of the facility). For example, the system can generate a timeseries of facility maps such that, for each facility map, the facility map is labeled with a level of air circulation in each region of the facility map. Alternatively, the system can: simulate, based on a schematic of the HVAC system of the facility and HVAC data (e.g., fan, heating, and cooling settings), air movement through the facility; estimate the direction and/or speed of air movement through the facility concurrent with each facility map in the timeseries of facility maps; and label the facility map with the direction and/or speed data generated by the simulation.

In yet another implementation, the system can generate a timeseries of facility maps such that each facility map represents the location of walls, windows, doors, or other fixed features of the facility within the facility map. Thus, the system can generate a time series of facility maps that is spatially representative of the facility.

### 9.1 Object Identification

Generally, the system identifies a set of objects in the timeseries of facility maps, the set of objects including a set of humans in Block S132 or in Block S232. More specifically, the system can execute an object classification model such as the object classification model described in U.S. patent application Ser. No. 16/845,525. The system can identify any type of object (animate or inanimate) or feature commonly found within the facility. In one implementation, the system can identify both the classification or type of object and its orientation for both humans and other objects as is further described below.

### 9.1.1 Human Identification

Generally, the system can analyze each image (or an aggregated image based on multiple overlapping images in the set of images) captured by the set of sensor blocks to detect humans located in various regions within the facility, and interaction distances between them. More specifically, the system can execute the methods described in U.S. patent application Ser. No. 16/845,525 in order to identify a position and/or orientation of a human within an image recorded by a sensor block.

In another implementation, the system can train an artificial neural network, as described in U.S. patent application Ser. No. 16/845,525 in order to detect a personal protection equipment (hereinafter “PPE”) status of a human detected in an image. For example, the system can detect whether a human detected in an image is wearing a mask based on the image.

Upon detecting a human within an image recorded by a sensor block of the system, the system can define a centroid or bounding box corresponding to the human representing the human's position within the image.

### 9.1.2 Human Effect Identification

Generally, the system can identify a set of objects in the set of images captured by the set of sensor blocks within a reporting period, the set of objects including human effects commonly used by humans in the facility, as described in U.S. patent application Ser. No. 16/845,525. For example, the system can identify human effects including: personal items; laptop computers; tablet computers; smartphones; keyboards; electronic mice; charging cables; data transfer cables; beverage containers; food containers; utensils; tissue paper; napkins; pairs of headphones or earphones; articles of clothing; wearable accessories; keys and/or keychains; wallets; pens; pencils; books; booklets; notebooks; and/or pieces of loose paper. Thus, the system can identify human effects, which may act as vectors of extant disease in the facility. However, the system may or may not distinguish (e.g., via separate classifications) between each of the aforementioned categories of human assets and, in one implementation, can group subsets of these human effects into broader classifications.

### 9.1.3 Asset Identification

In addition to detecting humans and human effects within an image recorded by a sensor block, the system can also identify assets of the facility such as seats, chairs, desks, desktop computers, monitors, whiteboards, conference tables, cubicle dividers, screens, plastic or glass shields, or any other common object deployed within the facility, as described in U.S. patent application Ser. No. 16/845,525. More specifically, the system can identify a set of objects in the timeseries of facility maps, the set of objects including: the set of humans; and a set of obstructions. Thus, the system can identify obstructions or objects that might block aerosolized droplets, larger droplets, or other disease vectors from transmission between humans in the workplace.

### 9.2 Video Tracking and Path Identification

In one implementation in which the set of sensor blocks are configured to capture the set of images at a high frame rate (e.g., greater than one frame-per-second), the system can track humans and/or other objects by identifying these humans and/or objects in consecutive frames, as described in U.S. Provisional Patent Application No. 63/197,922; and generating paths representing the movement of the human or other object through the facility. Thus, the system can detect human movement through the same region of a facility (e.g., a hallway) during temporally separated instances within the reporting period and identify this event as a potential contact event or, additionally or alternatively, directly include the path information extracted from the set of video feeds in a transmission feature vector for the reporting period, as is further described below.

### 10. Transmission Feature Vector

Generally, the system can generate a transmission feature vector based on the set of objects in the timeseries of facility maps and the set of extant disease metrics associated with the reporting period in Block S140. Additionally, the system can generate a similar training transmission feature vector based on the training set of objects in the training timeseries of facility maps and the training set of extant disease metrics associated with a sampling period in Block S240. More specifically, the system can generate a training feature vector that defines the state of humans and objects in the facility concurrent with each facility map in the timeseries of facility maps. Thus, the system incorporates the spatial-temporal data for humans and objects in the facility and the state of the set of extant diseases that are likely present with the facility into the transmission feature vector in order to input these data into the facility health model, as described below.

In one implementation, the system can reduce noise in the transmission feature vector and, therefore, noise in the predictions of health metrics and generation of mitigation responses, by executing preprocessing based on the timeseries of facility maps and the set of extant disease metrics to isolate potential contact events during which transmission was likely to occur between humans in the facility. Examples of this implementation are further described below. Alternatively, the system can generate a feature vector including the raw (i.e., unprocessed) timeseries of facility maps and the set of extant disease metrics.

### 10.1 Social Distance Calculation

In one implementation, the system can generate a transmission feature vector that includes, for each human detected in the facility during the reporting period or sampling period, a social distance score representing the cumulative social distancing practice by the human during the reporting period. More specifically, the system can, for each facility map in the timeseries of facility maps and for each human in the set of humans identified in the facility map: estimate a set of social distances between the human and each other human in a local subset of other humans in the set of humans, the local subset of other humans located in a room of the facility with the human based on the facility map; calculate a social distance score for the human based on the set of social distances; and aggregate the social distance score into the transmission feature vector. Thus, the system can generate a transmission feature vector that enables correlation between the cumulative social distance practice by each human in the facility and subsequent health outcomes of these humans via the facility health model.

In one implementation, upon detecting a pair of humans within the same room or region of a facility map in the timeseries of facility maps, the system can calculate the social distance between these humans. More specifically, the system can: extract from the facility map a reference position for each human in the pair of humans (e.g., a centroid, or a point on a bounding box) and a height associated with these reference positions in three-dimensional space (e.g., estimated via photogrammetric techniques in Block S130 or Block S230; and calculate the distance between these reference positions. Thus, the system can utilize photogrammetric techniques to convert pixel locations in the set of images, which may be distorted by the lens of the sensor block, to an accurate position in three-dimensional space in Blocks S130 and S230, prior to calculating a specific distance between the humans detected in the image.

In another implementation, the system can access ceiling height data corresponding to a sensor block that recorded an image in the set of images depicting the pair of humans in order to estimate the height of the pair of humans within the image and more accurately calculate the height of reference positions for these humans in three-dimensional space in Blocks S130 and S230. Alternatively, the system can estimate the ceiling height of the floor area based on the set of images and known dimensions of objects depicted therein. Alternatively, the system can incorporate these bounding boxes and three-dimensional positions into each facility map in the timeseries of facility maps in advance of calculating the social distancing score for each human in the timeseries of facility maps.

In another implementation, the system can detect whether each human detected in the timeseries of facility maps is sitting or standing in Blocks S130 and S230 and can access demographic height data to estimate the height of each human's head within the facility in either the sitting or the standing position. Thus, the system can indicate the position of each human's head in each facility map in the timeseries of facility maps without identifying the human or estimating the height of the human based on the image and define the position of each human's head in the image as a reference position for the human. In one example, the system can also calculate the orientation of a human's face in order to weigh distances calculated between a pair of humans based on whether these humans are facing each other or facing away from each other.

The system can, for each human detected in a facility map in the timeseries of facility maps: indicate a centroid of pixels comprising the depiction of the human in the image from which the system generated the facility map; define this centroid as the reference position of the human in each facility map; and calculate distances between the human and other humans in the image based on these reference positions.

Alternatively, the system can: calculate a bounding box encompassing the depiction of the human based on the image from which the facility map generated; locate a point on bounding box closest to the second human in the pair of humans; and define this point as reference position. Because the bounding box surrounds the pixels identified as a human in the image, the system can assume that the reference position in the facility map depicts a point on the floor of the facility.

In one implementation, the system records only the distance between each human and the closest other human depicted in a facility map in the set of facility maps. Alternatively, the system calculates and records the distance between each pair of humans in the facility map. For example, in response to identifying three humans within the facility map, a first human, a second human, and a third human, the system can calculate and record a first distance between the first human and the second human, a second distance between the first human and the third human, and a third distance between the second human and the third human, independent of the position of these humans relative to each other. Thus, the system can more accurately represent disease transmission hazards caused by closely spaced groups within the facility by recording these events as multiple social distances.

The system can also record other social distancing metrics such as human density for each facility map in the timeseries of facility maps. For example, the system can; access a floorplan to determine the area of a region in the facility map or can estimate the area of this region; and divide the number of humans detected in the image by the floor area.

### 10.1.1 Obstruction Correction

In one implementation, as described above, the system can identify obstructions (e.g., cubicle walls, room dividers) within the facility, which may affect the detected distance between a pair of humans and correct the calculated distance between this pair of humans. More specifically, the system can: identify obstructions within an image recorded by a sensor block of the system; detect whether shortest paths between humans in the image are intersected by the identified obstructions; and correct the distance calculations for this pair of humans to account for the obstructions by increasing the social distance calculation for the pair of humans. For example, the system can correct distances based on the presence of intervening obstructions by calculating the geodesic distance around these intervening obstructions or by increasing the social distance by a constant correction based on the type of obstruction between the pair of humans. Thus, the system can account for and reduce the frequency of calculating incorrect social distances over obstacles identified in each facility map in the timeseries of facility maps.

The system can then detect whether direct lines between reference positions calculated for humans within each facility map intersect with the identified obstructions. For example, the system, upon calculating a distance between a pair of humans detected in the facility map, can calculate a vector in three- or two-dimensional space between the reference positions representing this pair of humans. The system can then represent the obstruction as a floor-to-ceiling wall and detect whether this wall intersects with the calculated vector representing the distance between the pair of humans. Alternatively, the system can detect intersections in two dimensions to reduce computational complexity. In one example, the system can estimate the height of the obstruction and represent the obstructions as a wall characterized by the estimated height of the obstructions. For example, the system can identify an obstruction as a cubicle wall and access a lookup table indicating the height of this cubicle wall. The system can then represent the cubicle wall in three-dimensional space at the indicated height. In this example, the system can correct distances for humans detected as being in a seated position while maintaining distances calculated between humans detected in a standing position as these humans may stand above the maximum height of the cubicle wall.

In one implementation, the system can: in response to detecting an obstruction along the direct line between the first human in the pair of humans and the second human in the pair of humans, exclude the detected interaction between the pair of humans. Thus, the system can assume that the obstruction indicates that no transmission of disease occurred between this pair of humans and can omit this distance from the set of social distances included in the social distance score for each human.

In another implementation, the system can: in response to detecting an obstruction along the direct line between the first human in the pair of humans and the second human in the pair of humans, calculate a corrected distance based on the shortest path between the pair of humans circumventing the detected obstruction. Thus, the system can maintain a record of a distance between a pair of humans separated by an obstruction but adjust this distance to indicate a reduced risk of transmission between this pair of humans.

### 10.1.2 Social Distancing Score

In one variation, the system can calculate a social distancing score for interactions between pairs of humans detected across multiple successive facility maps in the timeseries of facility maps. More specifically, the system can: detect a first pair of humans in a first facility map in the time series of facility maps; detect a second pair of humans in a second facility map in the timeseries of facility maps; associate the second pair of humans with the first pair of humans based on the relative positions of the first pair of humans in the first facility map and the second pair of humans in the second facility map; estimate a duration of exposure for this pair of humans based on the intervening time between the first facility map and the second facility map; and calculate a social distancing score for the interaction of this pair of humans based on the duration of exposure and the distance between the pair of humans in the first facility map and in the second facility map. Thus, the system can detect interactions between humans that continue across multiple images without uniquely identifying humans within the facility.

In one implementation, the system can associate a first human detected in a first facility map with a second human detected in a second facility map by detecting that a second reference position calculated for the second human is located within a threshold distance of a first reference position calculated for the first human. Thus, the system can, in response to detecting a human in close to the same position as a human detected in a prior facility map, assume that this human is the same individual and that any interaction between this human and other humans depicted in the facility maps have occurred for the intervening time between facility maps (i.e., the sampling period of the sensor block).

For example, the system can: detect a first pair of humans in a first facility map and a second pair of humans in a second facility map in approximately the same position (e.g., each human in the second pair of humans is within a threshold distance of a human in the first pair of humans); and calculate an exposure duration of ten minutes based on a ten minute sampling interval between the first facility map and the second facility map.

In another implementation, the system can calculate a social distancing score based on an average distance for an interaction between a pair of humans (e.g., across multiple consecutive facility maps) and the duration of the exposure. For example, the system can calculate a social distancing score by multiplying the average distance for an interaction between a pair of humans with the estimated duration of the interaction of the pair of humans. Alternatively, the system can integrate the exposure time and an inverse distance between a pair of humans over multiple facility maps. For example, if the system detects that a pair of humans are five feet apart for ten minutes and ten feet apart for thirty minutes based on multiple successive facility maps, the system can calculate a social distancing score of 5 (equal to ten divided by five plus thirty divided by ten).

In yet another implementation, the system can anonymously identify humans (e.g., by assigning a unique anonymous identifier to each human) based on visual characteristics (represented as a classification vector) extracted based on the set of images from which the system generated each facility map. The system can then track each human and calculate a social distancing score for each human based on the interactions involving the human detected at any sensor block in the set of sensor blocks deployed to the facility. Thus, the system can more accurately determine the duration of interactions between humans within the facility by tracking specific humans across the fields of view of multiple sensor blocks in the facility.

Upon calculation of a social distancing score for each human in the set of humans, the system can generate the transmission feature vector by including within the feature vector the social distancing score of each human in the set of humans identified in the timeseries of facility maps for the reporting period.

### 10.2 Contact Events

In one implementation, the system can generate the transmission feature vector based on the detection of contact events between humans in the timeseries of facility maps. More specifically, the system can, for each extant disease in a set of extant diseases identified based on the set of extant disease metrics, generate the transmission feature vector by: detecting a set of contact events during the reporting period based on the set of objects in the timeseries of facility maps and the set of extant disease metrics; and calculating a set of contact events based on the set of contact events; and aggregating the set of contact events into the transmission feature vector. Thus, by identifying contact events, as defined by epidemiological standards associated with particular disease or disease types, the system can better apply extant disease metrics such as the mode of transmission and probability of transmission to predict the transmission of disease within the facility.

The system can define contact events based on epidemiological standards applicable to the mode of transmission of each extant disease identified in the set of extant disease metrics. For example, the system can define a first threshold distance corresponding to a direct contact event, a second threshold distance corresponding to a droplet spread contact event, and a third threshold distance corresponding to an airborne spread contact event. Thus, the system can identify or classify the type of contact event based on the distance between a first human and a second human identified in a facility map in the timeseries of facility maps.

Additionally or alternatively, the system can associate a non-distance-related set of criteria for detection of particular types of contact events. For example, the system can identify a vehicle-born or surface borne contact event, if an asset or other object identified in the facility map is touched by a first human at a first time and a second human at a second time within a threshold duration of the first time. In another example, the system can, based on accessed HVAC-based air circulation patterns in the facility, identify an airborne contact event upon detecting air circulation across two humans identified in the facility map. Therefore, based on the mode of transmission of each extant disease in the set of extant diseases, the system can associate identified contact events with applicable extant diseases via the facility health model.

In one implementation, the system can associate additional metadata characterizing each contact event in the set of contact events and include this metadata in the transmission feature vector. For example, the system can, for each extant disease in the set of extant diseases: calculate a duration of each contact event in the set of contact events based on the set of objects in the timeseries of facility maps and the set of extant disease metrics; and aggregate the duration of each contact event in the set of contact events into the transmission feature vector. In another example, the system can, for each extant disease in the set of extant diseases: classify each contact event in the set of contact events based on the set of objects in the timeseries of facility maps and the set of extant disease metrics; and aggregated these classified contact events into the transmission feature vector.

Thus, the system can generate a feature vector that represents: a set of contact events detected based on the timeseries of facility maps, the duration of each contact event, the type or classification of each contact event, the location in the facility at which the contact event occurred and/or any other relevant metadata characterizing the contact event; and the set of extant disease metrics.

### 10.3 Simulation

In one implementation, the system can generate a transmission feature vector representing the output of a simulation of disease transmission in the facility. More specifically, for each extant disease in a set of extant diseases identified based on the set of extant disease metrics, the system can generate the transmission feature vector by: simulating transmission of the extant disease within the facility based on the set of objects in the timeseries of facility maps and the set of extant disease metrics to estimate a number of newly infected humans in the facility and a reproduction rate of the extant disease in the facility; and aggregating the number of newly infected humans and the reproduction rate of the extant disease into the transmission feature vector. Thus, the system can simulate disease transmission based on the characteristics of the set of extant diseases (including their prevalence within the facility) and the temporospatial data represented by the time series of facility maps associated with the reporting period, thereby providing an accurate signal for health outcome prediction via the facility health model.

In one implementation, the system can simulate disease transmission within the facility by randomly designating a proportion of humans identified in the timeseries of facility maps as being infected with an extant disease in the set of extant diseases based on an estimated prevalence of the disease at the facility. Additionally, the system can randomly designate a proportion of humans identified in the timeseries of facility maps as being vaccinated for the extant disease, if a vaccine exists for the extant disease. The system can then identify contact events involving these humans and identify a transmission event based on the probability of transmission of the extant disease, the duration of the contact event, and/or the type of the contact event. The system can continue this simulation for each facility map in the timeseries of facility maps until the end of the reporting period or sampling period. Thus, by executing this simulation for each extant disease in the set of extant diseases, the system can generate a number of new infections for each extant disease in the set of extant diseases.

In another implementation, the system can execute the above-described simulation multiple times, designating a different subset of humans in the set of humans as infected (or vaccinated) during each simulation. Thus, based on the movements of the infected humans in the facility, the system may output different simulation results based on these random designations, thereby generating a distribution of new infections and a distribution of reproduction numbers for each extant disease representing possible disease transmission outcomes for the facility. The system can then generate a transmission feature vector including the distribution of new infections and/or the distribution of reproduction numbers for each extant disease in the set of extant diseases.

In yet another implementation, the system can generate a transmission feature vector including a heatmap indicating the location within the facility of each simulated disease transmission, thereby providing additional data with which to prompt a mitigation response to reduce disease transmission within the facility.

### 11. Facility Health Model

Generally, the system can calculate a predicted timeseries of health metrics for the facility based on the transmission feature vector for a reporting period and a facility health model in Block S150. More specifically, the system can execute a facility health model—such as an artificial neural network, long short-term memory model, or any other machine learning model—configured to relate an input transmission feature vector representing a reporting period to a timeseries of health metrics for a time period subsequent to the reporting period. For example, the system can, via the facility health model, generate a timeseries of estimated attendance of the set of humans at the facility spanning the three weeks subsequent to the reporting period. Thus, the system executes a facility health model capable of long-range and temporally precise predictions of health metrics characterizing the set of humans within the facility.

In one implementation, the system executes a generalized facility health model trained based on training examples derived from many different facilities. This generalized facility health model may be robust to the effects of over training but may output less relevant health metrics to the particular facility, since health metrics of interest may vary between facilities. Alternatively, the system can execute a facility-specific model trained based on training examples derived from a specific facility such that each training example includes a training feature vector derived from the facility and a timeseries of facility-relevant health metrics. Thus, the system can train a facility health model configured to accurately predict a set of health metrics relevant to a particular facility that may account for particular characteristics of the facility, which cannot be represented by the feature vector alone.

### 11.1 Training

As shown in FIG. 2, by executing Blocks of the method S200, the system can train a facility health model for execution in Block S150 of the method S100. More specifically, the system can: access a training set of extant disease metrics associated with the sampling period in Block S210; access a training set of images of the facility captured during the sampling period by a set of sensor blocks deployed in the facility in Block S220; aggregate the training set of images into a training timeseries of facility maps depicting the facility during the sampling period in Block S230; identify a training set of objects in the timeseries of facility maps, the training set of objects comprising a training set of humans in Block S232; generate a training transmission feature vector based on the training set of objects in the training timeseries of facility maps and the training set of extant disease metrics associated with the sampling period, the training transmission feature vector in Block S240; and access a timeseries of health metrics for a causal period subsequent to the sampling period in Block S250. The method S200 also includes training the facility health model based on: the training transmission feature vector for each sampling period in the training period; and the timeseries of health metrics for the causal period subsequent to each sampling period in the training period in Block S260. Thus, the system: generates a set of training examples, each including a training transmission feature vector and a set of health metrics for a causal period associated with and subsequent to the sampling period; and trains the facility health model based on the set of training examples via a supervised learning algorithm.

Generally, Blocks S210, S220, S230, S232, and S240 correspond to Blocks S110, S120, S130, S132, and S140 respectively. Thus, the implementations described above with respect to Blocks S110, S120, S130, S132, and S140 are applicable to Blocks of S210, S220, S230, S232, and S240.

In Block S250, to complete a set of training examples for the facility health model, the system can access, from a database, a set of health metrics representing the health of humans at the facility during a causal period subsequent to the sampling period.

In Block S260, the system can then execute a supervised training algorithm based on the set of training examples in order to train the facility health model to predict the set of health metrics given a transmission feature vector.

In one implementation, the system can continually retrain the facility health model based on transmission feature vectors generated for reporting periods (as opposed to training periods) during execution of the method S100. In this implementation, the system can also include mitigation responses output by the system or designated by an administrator of the facility in order to account for typical mitigation responses in the output of the facility health model. Thus, in this implementation, via the facility health model, the system can predict the specific improvement to the timeseries of health metrics correlated with each mitigation response in a set of possible mitigation responses.

### 11.2 Output Health Metrics

Generally, the system can train a facility health model to output a timeseries of any health metric or combination of health metrics characterizing the health of the population of humans in the facility. Alternatively, the system can train a facility health model to output a timeseries of a health metric that may be proxy for the health of a human in the facility (e.g., a productivity metric or behavioral metric, which may indirectly indicate the health of the population of humans in the facility). Thus, when executing Block S150 of the method S100, the system can generate actionable timeseries of health metrics to drive mitigation responses and/or adjustments by the administrators of the facility.

In one implementation, the system can predict a timeseries of symptomatic humans in the facility for a causal period subsequent to the reporting period based on the transmission feature vector and the facility health model. For example, the system can indicate a number or proportion of humans in the facility that are predicted to be experiencing symptoms of each extant disease in the set of extant diseases. Thus, the system can trigger mitigation responses in response to detecting that the number of humans experiencing symptoms exceeds a predetermined threshold or meets any other criteria. Additionally, in this implementation, based on known symptoms of the set of extant diseases indicated by the set of extant disease metrics, the system can predict the number or proportion of humans that are functionally incapacitated, will not attend the facility, or will be otherwise impaired.

In another implementation, the system can predict a timeseries of lost man-hours for a causal period subsequent to the reporting period based on the transmission feature vector and the facility health model. For example, the system can predict a timeseries of sick days accrued for a set of humans employed at the facility over a month subsequent to the reporting period based on a transmission feature vector for the reporting period. Thus, the system can initiate mitigation responses in preparation for a temporary reduction in the size of a workforce at the facility.

In yet another implementation, the system can predict a timeseries of lost productivity for a causal period subsequent to the reporting period based on the transmission feature vector and the facility health model. In this implementation, the system can predict any measure of lost productivity applicable to the facility such as net revenue or profit, total unit production rate, or progress toward a particular goal or a particular project (e.g., project days lost or days behind). Thus, the system can indirectly predict the health of humans at the facility based on the effect that the health of these humans will have on their productivity.

### 12. Mitigation Response

Generally, the system can prompt a mitigation response at the facility based on the predicted timeseries of health metrics in Block S160. Initially, the system can prompt mitigation responses based on a set of triggers, threshold, or other criteria for the timeseries of health metrics. For example, the system can reference a set of mitigation response criteria and automatically prompt mitigation responses based on whether the timeseries of health metrics predicted by the facility health model satisfy any subset of these mitigation response criteria. However, as additional mitigation responses are prompted by the system or designated by an administrator of the facility, the system can retrain the facility health model, as described above, in response to these mitigation responses. The system can then leverage this facility health model that accounts for mitigation responses to test the effect of each mitigation response in a set of possible mitigation responses and select the mitigation response that has the most positive effect on the predicted timeseries of health metrics according to the facility health model.

For example, for a particular reporting period, the system can: generate a first transmission feature vector based on the timeseries of facility maps for the reporting period the set of extant disease metrics for the reporting period, and a first potential mitigation response for the reporting period; and generate a second transmission feature vector based on the timeseries of facility maps for the reporting period the set of extant disease metrics for the reporting period, and a second potential mitigation response for the reporting period. The system can then predict a first timeseries of health metrics based on the first transmission feature vector and a second timeseries of health metrics based on the second transmission feature vector; and prompt either the first mitigation response or the second mitigation response based on the mitigation response that resulted in the greatest improvement in health outcomes based on the first timeseries of health metrics and the second timeseries of health metrics. Thus, the system can leverage predictions of the facility health model to select a mitigation response to automatically prompt at the facility.

### 12.1 Types of Mitigation Responses

In one implementation, the system can prompt a mitigation response at the facility by automatically adjusting a work-from-home schedule to reduce attendance at the facility based on the predicted timeseries of health metrics. Thus, the system can reduce the density of humans within the facility for subsequent days following the reporting period.

In another implementation, the system can prompt a mitigation response at the facility by automatically reducing a maximum occupancy of conference rooms within the facility based on the predicted timeseries of health metrics. Thus, the system can reduce the number of people gathering within small spaces at the facility, thereby reducing the number of contact events occurring at the facility.

In yet another implementation, the system can prompt a mitigation response at the facility by automatically reducing a maximum occupancy of an agile desk area within the facility based on the predicted timeseries of health metrics. For example, the system can automatically designate (via a desk reservation or scheduling system), a proportion or number of desks within an agile desk area that may not be reserved by humans at the facility. Additionally, the system can select the specific location of these excluded desks to minimize the probability of contact events occurring in the facility.

In yet another implementation, the system can prompt a mitigation response by adjusting a social distancing policy at the facility to increase the minimum allowable distance between humans in the facility. Thus, assuming compliance with the social distancing policy within the facility, the system can reduce the probability of contact events occurring by directing humans in the facility to increase the distance between themselves and others.

In yet another implementation, the system can prompt a mitigation response by adjusting a personal protection equipment (hereinafter, “PPE”) policy at the facility. For example, the system can update the PPE policy to include mandatory mask wearing by all humans in the facility. Thus, assuming compliance with the PPE policy, the system can reduce disease transmission in the facility by directly blocking viral or bacterial particles via additional PPE.

In yet another implementation, the system can prompt a mitigation response by: accessing a heatmap of contact events or simulated disease transmission within the facility included in the transmission feature vector for a reporting period; identifying a cluster of disease transmission in a region of the facility based on the heatmap of contact events or simulated disease transmission; and automatically restricting access to the region encompassing the cluster of contact events or simulated disease transmission in the facility. For example, the system can detect, via a heatmap of contact events, that a cluster of contact events is likely to have occurred in a breakroom at the facility. In response, the system can automatically restrict access to the breakroom via an associated scheduling or security system of the facility.

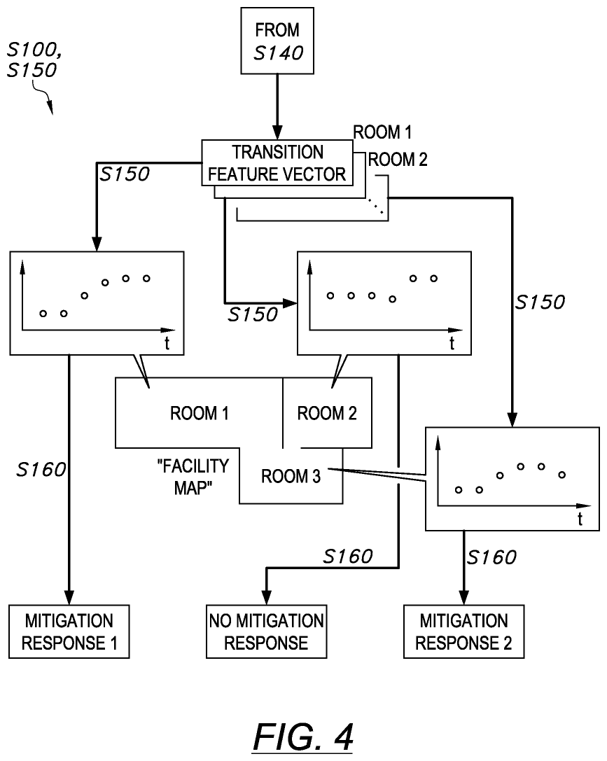

As shown in FIG. 4, the system can prompt localized mitigation response by: calculating a set of predicted timeseries of health metrics for each region of the facility based on the transmission feature vector and the facility health model; and prompting a distinct mitigation response for each region of the facility based on the set of predicted timeseries of health metrics. Thus, the system can separately prompt the most effective mitigation response for each region of the facility based on the local spatial context of each region of the facility.

### 13. Administrative Portal

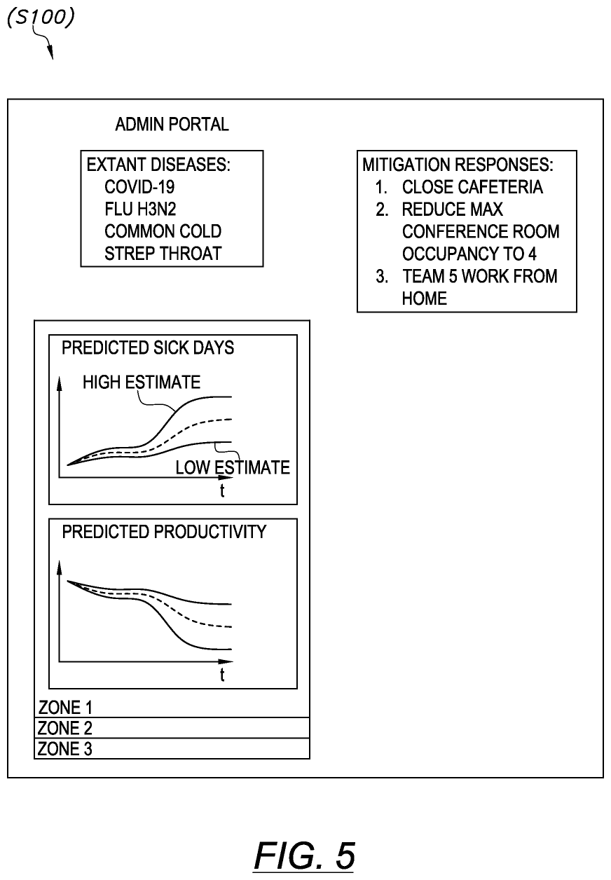

As shown in FIG. 5, the system can report the timeseries of health metrics and the mitigation response at an administrator portal of the facility. More specifically, upon predicting a set of health metrics for the facility and prompting a mitigation response, the system can render these metrics via an administrative portal or application, which may be viewed by an administrator or other user of the system. In particular, the system can: render a representation of the predicted timeseries of health metrics for a most recent reporting period; and render an alert recommending the prompted mitigation response or rendering a representation of the automatically prompted mitigation response. Thus, the system can indirectly effect mitigation responses or changes to the management of the facility by notifying administrators of disease transmission and predicted health outcomes for humans in the facility.

### 14. Monitoring Social Distancing Policy Adherence

In addition to prompting mitigation responses, the system can also monitor social distancing policy adherence within the facility based on the set of images captured by the set of sensor blocks deployed in the facility. Generally, the system can access a social distancing policy that assigns a minimum allowable distance for a detected interaction between humans within the facility to contexts within the facility. More specifically, the system can access a social distancing policy that designates a particular context (e.g., a floor area within the facility, an interaction duration, a status of humans within the facility, a cumulative social distancing score for a floor area within the facility) and assigns a particular minimum allowable distance to the particular context. Thus, the system can track compliance with more complex social distancing policies that specify differing minimum allowable distances based on the context of an interaction between humans.

In one implementation, the system accesses a social distancing policy specifying a single minimum allowable distance for all contexts. For example, the system can access a social distancing policy specifying a six-foot minimum allowable distance for all floor areas within the facility.

In another implementation, the system accesses a social distancing policy specifying a first minimum allowable distance corresponding to a first floor area within the facility and a second minimum allowable distance corresponding to a second floor area. Additionally, the system can access a social distancing policy that specifies a minimum allowable distance for each type of floor area within the facility. For example, the system can access a social distancing policy specifying a six-foot minimum allowable distance within an agile desk area and an eight-foot minimum allowable distance within a conference room. Thus, the system can access social distancing policies that reflect the increased risk of exposure to communicable diseases caused by difference in the type of environment.

In yet another implementation, the system can access a social distancing policy that specifies a minimum allowable distance for a floor area as a function of the air volume within the area. In this implementation, the system can characterize each sensor block based on a ceiling height at the location of the sensor block (via automatic detection or labelling upon deployment within the facility). In one example, the system can calculate a volume of a room based on the ceiling height for the sensor block and the area of the floor area (e.g., based on a floorplan or calculated based on images recorded by the set of sensor blocks). Subsequently, the system can access a social distancing policy based on the calculated air volume within the room. In another example, the system can access heating ventilation and air conditioning information associated with a floor area in order to select a social distancing policy corresponding to the level of ventilation within a floor area. The system can access HVAC information such as the number of vents leading into and out of the floor area, the relative air pressure within the floor area, the filter type for air conditioners or heaters venting into the floor area, whether the floor area is open to the outdoors, or any other HVAC related information associated with the floor area.

In yet another implementation, the system accesses a social distancing policy specifying a first minimum allowable distance corresponding to a first interaction duration and a second minimum allowable distance corresponding to a second interaction duration. For example, the system can access a social distancing policy specifying that interactions lasting ten minutes or longer are assigned a twelve-foot minimum allowable distance while interactions lasting less than ten minutes are assigned a six-foot minimum allowable distance. Thus, the system can access social distancing policies that reflect increased risk of transmission during long term social interaction.

In yet another implementation, the system accesses a social distancing policy specifying a first minimum allowable distance corresponding to a status of a first human and a second minimum allowable distance corresponding to a status of a second human. For example, the system can access a social distancing policy that specifies a six-foot minimum allowable distance for humans that are detected wearing a mask and specifies a twelve-foot minimum allowable distance for humans that are detected without a mask. Thus, the system can access social distancing policies that account for differences in PPE worn by humans within the facility.

In yet another implementation, the system accesses a social distancing policy specifying a maximum allowable human density within a floor area. For example, the system can access a social distancing policy specifying a maximum allowable human density less than one human per 100 square feet. Thus, the system can evaluate compliance with other goal metrics associated with reduction in transmission of communicable diseases.

The system can access a social distancing policy that specifies minimum allowable distances based on a standardized unit of measurement (e.g., feet, meters). Alternatively, the system can access a social distancing policy that specify minimum allowable distances relative to common objects within the facility (e.g., a single desk length). Thus, the system can reduce inaccuracies in distances calculated via photogrammetry by evaluating social distancing profile violations based on known dimensions of objects within the floor area.

In addition to accessing social distancing policies that specify minimum allowable distances between humans or maximum allowable human densities within the facility, the system can access social distancing policies that specify minimum allowable distances or maximum densities for assets within the facility. For example, the system can access a social distancing policy specifying a maximum density of chairs within a conference room or desks within an agile desk area or a minimum distance between these chairs.

Additionally, the system can access a social distancing policy specifying a maximum social distancing score, the maximum social distancing score representing a maximum acceptable likelihood of transmission of a disease within the floor area depicted in an image. For example, the system can calculate a maximum social distancing score based on the distance between a first human and other humans within the image and an estimated time spent by the human in proximity to other humans in the image. Thus, the system can detect violations based on a multivariable function of many social distancing metrics rather than a single social distancing metric.

### 14.1 Real-Time Alerts

In one implementation, the system can, in response to detecting an ongoing violation of the social distancing policy of the facility, the system can generate a real-time alert at an administrative portal of the facility indicating a type of the violation and/or the location of the violation within the facility. Thus, the system can direct administrators of the facility to inform humans within the facility of the social distancing policy, thereby reducing the likelihood of continued non-compliance with the social distancing policy.

Upon generating a real-time alert, the system can identify the location of the ongoing violation of the social distancing policy by referencing a lookup table indicating correspondence between sensor blocks and locations within the facility. Additionally, the system can indicate the type of violation of the social distancing policy. For example, the system can detect, based on a set of consecutive images, that a pair of humans are spending an extended period of time within the minimum allowable distance of one another and can indicate this violation as a proximity violation. In another example, the system can detect, based on a set of consecutive images, that the density of humans in the floorspace exceeds a maximum density and can indicate this violation as a density violation. Likewise, the system can detect and identify asset-related proximity or density violations.

In one implementation, the system generates a real-time alert; and renders the real-time alert at an administrative application executing on a computing device of an administrator of the facility (e.g., a smartphone application executing on a smartphone of an administrator of the facility). In another implementation, the system can include the image or images from which the violation of the social distancing policy was detected in the real-time alert.

In addition to generating real-time alerts for an administrator portal, the system can also generate real-time alerts for a custodial portal viewable by custodial personnel of the facility. For example, the system can detect a density violation within a particular floor area of the facility and, in response, can generate a real-time alert for a custodial portal to indicate that the floorspace needs a cleaning. Thus, by rendering real-time alerts for custodial personnel in addition to administrators, the system can better coordinate sanitation efforts within the facility to prevent the spread of communicable diseases.

Furthermore, the system can generate real-time alerts for portals distributed within the floorspace in order to notify the humans committing the violation of the violation in real-time. For example, in response to detecting a density violation within a conference room, the system can render a real-time notification at a display within the conference room to indicate that the current occupancy of the conference room exceeds the maximum occupancy of the conference room. Thus, the system can remind humans within the facility of ongoing violations, thereby reducing opportunities for communicable disease spread without requiring intervention by administrators or custodial personnel.

### 14.2 Long Term Guidance

Generally, the system can also generate long term guidance in the form of periodic summary reports, which can include multiple social distancing summary metrics and visual representations of these metrics. Thus, the system can generate a periodic summary indicating the effectiveness of current measures within the facilities to engender compliance with the social distancing policy for the facility.

In one implementation, the system can generate a summary report including a distribution of distances between pairs of humans detected by the system. In this implementation, the system can also classify peaks within the distribution of distances corresponding to various types of interactions within the facility. For example, the system can: identify a first peak as an inter-desk distance in the facility approximately (e.g., plus or minus twenty percent) equal to a standard distance between desks in the facility; and identify a second peak different from the first peak representing the peak interaction distance between a pair of humans within the facility.

In another implementation, the system can generate a summary report including a heatmap of the facility indicating each detected human within the facility during the time period represented by the summary report. Alternatively, the system can generate a summary report including a heatmap of the facility indicating each detected social distancing policy violation during the time period. Thus, a member of the custodial personnel or an administrator may view the heatmaps in the summary report and prioritize a specific region within a floor area of the facility for cleaning or for the addition of targeted measured to prevent transmission of communicable disease.

### 14.2 Automatic Responses

Generally, the system can automatically respond to a set of non-compliance criteria, via a cooperating scheduler or asset manager application, for example, in order to increase compliance with the social distancing policy prior to direct intervention by administrators or custodial personnel. More specifically, the system can: detect a set of non-compliance criteria; and, in response to detecting the non-compliance criteria, update a scheduler, an asset manager, and/or the social distancing policy itself corresponding to the detected non-compliance criteria.

In one implementation, the system can: upon detecting a threshold proportion of interactions between humans in the facility at a distance less than the minimum allowable distance, update the social distancing policy to further increase the minimum allowable distance. For example, in response to detecting that greater than 25% of interactions between humans within the facility are occurring at less than the minimum allowable distance of six feet, the system can update the social distancing policy by increasing the minimum allowable distance to eight feet, thereby decreasing the frequency of interactions occurring at less than six feet.

In another implementation, the system can, in response to detecting greater than the maximum allowable human density within a floor area, automatically reschedule meetings, events, reservations, or the like scheduled to occur with the floor area to a different floor area of the facility (or in an alternate facility) in order to provide time for viral particles or bacteria to diminish or be removed from the area prior to humans continuing to occupy the area.

In yet another implementation, the system can, in response to detecting greater than a maximum asset density within a floor area of the facility or less than a minimum allowable asset distance between two assets within the facility, automatically generate a work order to relocate the assets in order to comply with the social distancing policy. For example, upon detecting that chairs within an agile desk area are spaced more closely than the minimum allowable distance between chairs as specified by the social distancing policy, the system can automatically generate a work order to move the chairs farther apart within the agile desk area. Thus, the system can more efficiently adapt the environment of the facility in order to prevent transmission of communicable diseases within the facility.

The systems and methods described herein can be embodied and/or implemented at least in part as a machine configured to receive a computer-readable medium storing computer-readable instructions. The instructions can be executed by computer-executable components integrated with the application, applet, host, server, network, website, communication service, communication interface, hardware/firmware/software elements of a user computer or mobile device, wristband, smartphone, or any suitable combination thereof. Other systems and methods of the embodiment can be embodied and/or implemented at least in part as a machine configured to receive a computer-readable medium storing computer-readable instructions. The instructions can be executed by computer-executable components integrated by computer-executable components integrated with apparatuses and networks of the type described above. The computer-readable medium can be stored on any suitable computer readable media such as RAMs, ROMs, flash memory, EEPROMs, optical devices (CD or DVD), hard drives, floppy drives, or any suitable device. The computer-executable component can be a processor but any suitable dedicated hardware device can (alternatively or additionally) execute the instructions.

As a person skilled in the art will recognize from the previous detailed description and from the figures and claims, modifications and changes can be made to the embodiments of the invention without departing from the scope of this invention as defined in the following claims.

I claim:

1. A method for mitigating disease transmission in a facility comprising:
during a training period:
for each sampling period in the training period:
accessing a training set of extant disease metrics associated with the sampling period;
accessing a training set of images of the facility captured during the sampling period by a set of sensor blocks deployed in the facility;
aggregating the training set of images into a training timeseries of facility maps depicting the facility during the sampling period;
identifying a training set of objects in the timeseries of facility maps, the training set of objects comprising a training set of humans;
generating a training transmission feature vector based on the training set of objects in the training timeseries of facility maps and the training set of extant disease metrics associated with the sampling period, the training transmission feature vector; and
accessing a timeseries of health metrics for a causal period subsequent to the sampling period; and
training a facility health model based on:
the training transmission feature vector for each sampling period in the training period; and
the timeseries of health metrics for the causal period subsequent to each sampling period in the training period; and
for a reporting period subsequent to the training period:
accessing a set of extant disease metrics associated with the reporting period;
accessing a set of images of the facility recorded during the reporting period by the set of sensor blocks;
aggregating the set of images into a timeseries of facility maps depicting the facility during the reporting period;
identifying a set of objects in the timeseries of facility maps, the set of objects comprising a set of humans;
generating a transmission feature vector based on the set of objects in the timeseries of facility maps and the set of extant disease metrics associated with the reporting period;
calculating a predicted timeseries of health metrics for the facility based on the transmission feature vector and the facility health model; and
prompting a mitigation response at the facility based on the predicted timeseries of health metrics.

2. A method for mitigating disease transmission in a facility comprising:
accessing a set of extant disease metrics associated with a reporting period;
accessing a set of images of the facility captured during the reporting period by a set of sensor blocks deployed in the facility;
aggregating the set of images into a timeseries of facility maps depicting the facility during the reporting period;
identifying a set of objects in the timeseries of facility maps, the set of objects comprising a set of humans;
generating a transmission feature vector based on the set of objects in the timeseries of facility maps and the set of extant disease metrics associated with the reporting period;
calculating a predicted timeseries of health metrics for the facility based on the transmission feature vector and a facility health model; and
prompting a mitigation response at the facility based on the predicted timeseries of health metrics.

3. The method of claim 2, wherein accessing the set of extant disease metrics comprises, for each disease in a set of diseases, accessing:
a prevalence;
a mode of transmission;
a probability of transmission; and
a health impact.

4. The method of claim 2, wherein accessing the set of images of the facility comprises:
at each sensor block in the set of sensor blocks and for each scan cycle during the reporting period:
capturing an image in the set of images, the image depicting the facility with a field of view of the sensor block; and
transmitting the image to a remote server; and
at the remote server, accessing the set of images.

5. The method of claim 2, wherein identifying the set of objects in the timeseries of facility maps comprises identifying the set of objects in the timeseries of facility maps and a location, in the facility, of each object in the set of objects.

6. The method of claim 2, wherein identifying the set of objects in the timeseries of facility maps comprises identifying the set of objects in the timeseries of facility maps, the set of objects comprising:
the set of humans; and
a set of obstructions.

7. The method of claim 2, wherein identifying the set of objects in the timeseries of facility maps comprises identifying the set of objects in the timeseries of facility maps, the set of objects comprising:
the set of humans; and
a set of human effects selected from a group consisting of:
a personal item;
a laptop computer;
a tablet computer;
a smartphone;
a keyboard;
an electronic mouse;
a charging cable;
a data transfer cable;
a beverage container;
a food container;
a utensil;
a tissue;
a napkin;
a pair of headphones;
an article of clothing;
a wearable accessory;
a key;
a keychain;
a wallet;
a pen;
a pencil;
a book;
a booklet;
a notebook; and
a piece of loose paper.

8. The method of claim 2, wherein generating the transmission feature vector comprises, for each facility map in the timeseries of facility maps, and for each human in the set of humans identified in the facility map:
estimating a set of social distances between the human and each other human in a local subset of other humans in the set of humans, the local subset of other humans located in a room of the facility with the human based on the facility map;
calculating a social distance score for the human based on the set of social distances; and
aggregating the social distance score into the transmission feature vector.

9. The method of claim 2, wherein generating the transmission feature vector comprises, for each extant disease in a set of extant diseases identified based on the set of extant disease metrics:
detecting a set of contact events during the reporting period based on the set of objects in the timeseries of facility maps and the set of extant disease metrics;
calculating a set of contact events based on the set of contact events; and
aggregating the set of contact events into the transmission feature vector.

10. The method of claim 9, wherein generating the transmission feature vector further comprises, for each extant disease in the set of extant diseases:
calculating a duration of each contact event in the set of contact events based on the set of objects in the timeseries of facility maps and the set of extant disease metrics; and
aggregating the duration of each contact event in the set of contact events into the transmission feature vector.

11. The method of claim 2, wherein generating the transmission feature vector comprises, for each extant disease in a set of extant diseases identified based on the set of extant disease metrics:
based on the set of objects in the timeseries of facility maps and the set of extant disease metrics, simulating transmission of the extant disease within the facility to estimate:
a number of newly infected humans in the facility; and
a reproduction rate of the extant disease in the facility; and
aggregating the number of newly infected humans and the reproduction rate of the extant disease into the transmission feature vector.

12. The method of claim 2, wherein calculating the predicted timeseries of health metrics for the facility comprises predicting a timeseries of symptomatic humans in the facility for a causal period subsequent to the reporting period based on the transmission feature vector and the facility health model.

13. The method of claim 2, wherein calculating the predicted timeseries of health metrics for the facility comprises predicting a timeseries of lost man-hours for a causal period subsequent to the reporting period based on the transmission feature vector and the facility health model.

14. The method of claim 2, wherein calculating the predicted timeseries of health metrics for the facility comprises predicting a timeseries of lost productivity for a causal period subsequent to the reporting period based on the transmission feature vector and the facility health model.

15. The method of claim 2, wherein prompting the mitigation response at the facility comprises adjusting a work-from-home schedule to reduce attendance at the facility based on the predicted timeseries of health metrics.

16. The method of claim 2, wherein prompting the mitigation response at the facility comprises reducing a maximum occupancy of conference rooms within the facility based on the predicted timeseries of health metrics.

17. The method of claim 2, wherein prompting the mitigation response at the facility comprises reducing a maximum occupancy of an agile desk area within the facility based on the predicted timeseries of health metrics.

18. The method of claim 2, further comprising reporting the timeseries of health metrics and the mitigation response at an administrator portal of the facility.

19. The method of claim 2:
wherein calculating the predicted timeseries of health metrics for the facility comprises calculating a set of predicted timeseries of health metrics for each region of the facility based on the transmission feature vector and the facility health model; and
wherein prompting the mitigation response at the facility comprises prompting a distinct mitigation response for each region of the facility based on the set of predicted timeseries of health metrics.

20. A method for mitigating disease transmission in a facility comprising:
accessing a set of extant disease metrics associated with a reporting period;
accessing a set of images of the facility recorded during the reporting period by a set of sensor blocks deployed in the facility;
generating a timeseries of facility maps depicting locations of a set of objects within the facility during the reporting period based on the set of images, the set of objects comprising a set of humans;
generating a transmission feature vector based on the set of objects in the timeseries of facility maps and the set of extant disease metrics associated with the reporting period;
calculating a predicted timeseries of health metrics for the facility based on the transmission feature vector and a facility health model; and
prompting a mitigation response at the facility based on the predicted timeseries of health metrics.
